(19) United States
(12) Patent Application Publication
(10) Pub. No.: US 2026/0108389 A1
(43) Pub. Date: Apr. 23, 2026

(54) SYSTEMS AND METHODS FOR DELIVERING AND DEPLOYING ADJUSTABLE SHUNTING SYSTEMS

(71) Applicant: Shifamed Holdings, LLC, Campbell, CA (US)

(72) Inventor: Eric Schultz, Los Altos, CA (US)

(21) Appl. No.: 19/148,308

(22) PCT Filed: Jan. 26, 2024
(86) PCT No.: PCT/US2024/013124

§ 371 Date: Jul. 15, 2025

Related U.S. Application Data
(60) Provisional application No. 63/481,926, filed on Jan. 27, 2023.

(51) Int. Cl. A61F 9/007; A61B 17/00; A61B 17/34

(52) U.S. Cl. CPC A61F 9/00781; A61B 2017/00367; A61B 17/3468

(57) ABSTRACT

The present technology is generally directed to shunt delivery systems and associated methods for delivering and deploying adjustable shunts. In some embodiments, the delivery systems include a housing, a pair of movement guides positioned within the housing, and an actuation assembly positioned at least partially between the pair of movement guides. The actuation assembly can include an actuator and a pair of arms operably engaged by the actuator. The actuation assembly can be configured to releasably carry a shunt at least partially between the pair of arms. The actuator can be movable relative to the pair of movement guides to cause movement of the pair of arms relative to the housing to, e.g., expose a length of the shunt distally beyond the arms.

### CROSS-REFERENCE TO RELATED APPLICATION(S)

This application claims priority to U.S. Provisional Patent Application No. 63/481,926, filed Jan. 27, 2023, and incorporated herein by reference in its entirety.

### TECHNICAL FIELD

The present technology generally relates to systems and methods for delivering and deploying implantable medical devices and, in particular, to delivery systems and associated methods for delivering and deploying intraocular shunts.

### BACKGROUND

Implantable shunting systems are widely used to treat a variety of patient conditions by shunting fluid from a first body region/cavity to a second body region/cavity. For example, shunting systems have been proposed for treating glaucoma. The flow of fluid through the shunting systems is primarily controlled by the pressure gradient across the shunt and the physical characteristics of the flow path defined through the shunt (e.g., the resistance of the shunt lumen). Conventional, early shunting systems (sometimes referred to as minimally invasive glaucoma surgery devices or “MIGS” devices) have shown clinical benefit; however, there is a need for improved shunting systems, systems for delivering such shunting systems, and techniques for addressing elevated intraocular pressure and risks associated with glaucoma. For example, there is a need for shunting systems capable of adjusting the therapy provided, including the flow rate between the two fluidly connected bodies. Further, there is a need for delivery systems for effectively and precisely delivering such shunting systems to target treatment locations within patients.

### BRIEF DESCRIPTION OF THE DRAWINGS

Many aspects of the present technology can be better understood with reference to the following drawings. The components in the drawings are not necessarily drawn to scale. Instead, emphasis is placed on illustrating clearly the principles of the present technology. Furthermore, components can be shown as transparent in certain views for clarity of illustration only and not to indicate that the component is necessarily transparent. Components may also be shown schematically.

FIGS. 1A and 1B are a perspective view and an exploded perspective view, respectively, of a delivery system configured for delivery of a shunting system in accordance with select embodiments of the present technology.

FIG. 1C is an exploded perspective view of an actuation assembly of the delivery system of FIG. 1A configured in accordance with embodiments of the present technology.

FIGS. 1D and 1E are perspective views illustrating operation of the actuation assembly of FIG. 1C in accordance with embodiments of the present technology.

FIGS. 2A and 2B are perspective views illustrating operation of the delivery system of FIG. 1A in accordance with embodiments of the present technology.

FIGS. 3A-3C are side cross-sectional views illustrating operation of the delivery system of FIG. 1A in accordance with embodiments of the present technology.

FIGS. 4A-4F illustrate movement of an actuator and arms of the delivery system of FIG. 1A in accordance with embodiments of the present technology.

FIGS. 5A-5E illustrate various stages of an operation for deploying a shunting system into a human eye using the delivery system of FIG. 1A in accordance with embodiments of the present technology.

### DETAILED DESCRIPTION

The present technology is directed to delivery systems and associated methods for delivering and deploying adjustable shunting systems. In some embodiments, the delivery systems include a pair of arms contained at least partially within and movable relative to a housing. The delivery system can be configured to carry a shunt, such as an adjustable shunting system, at least partially between the pair of arms. In some embodiments, the delivery system can include an actuator operably coupled to the pair of arms such that movement of the actuator can cause corresponding movement of the pair of arms, e.g., relative to the shunt. In at least some embodiments, for example, the actuator can be moved vertically to cause the pair of arms to open by, e.g., pivoting the pair of arms outwardly away from the shunt. Additionally, or alternatively, the actuator can be moved longitudinally relative to the housing to cause the pair of arms to move longitudinally to, e.g., increase or decrease a length of the portion of the shunt exposed beyond the pair of arms. During a procedure for, e.g., implanting the shunt in an eye of a patient, a user can manipulate the actuator to change a position of the pair of arms relative to the shunt. In at least some instances, for example, the user can increase a length of the portion of the shunt that is exposed beyond the pair of arms prior to inserting the shunt into the eye of the patient.

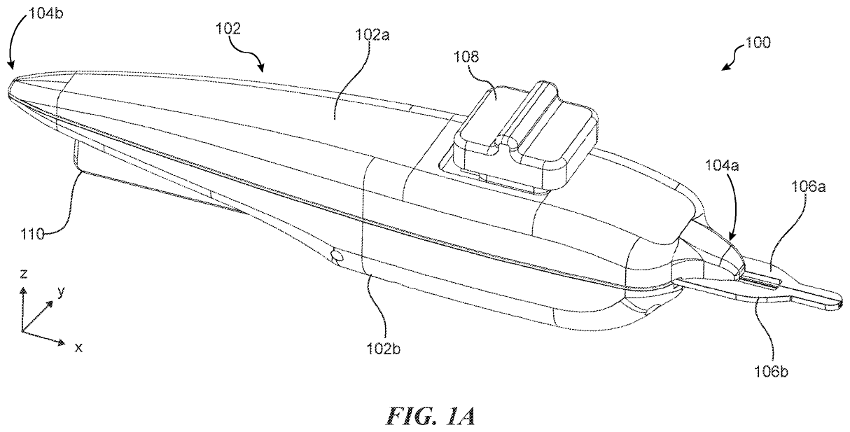

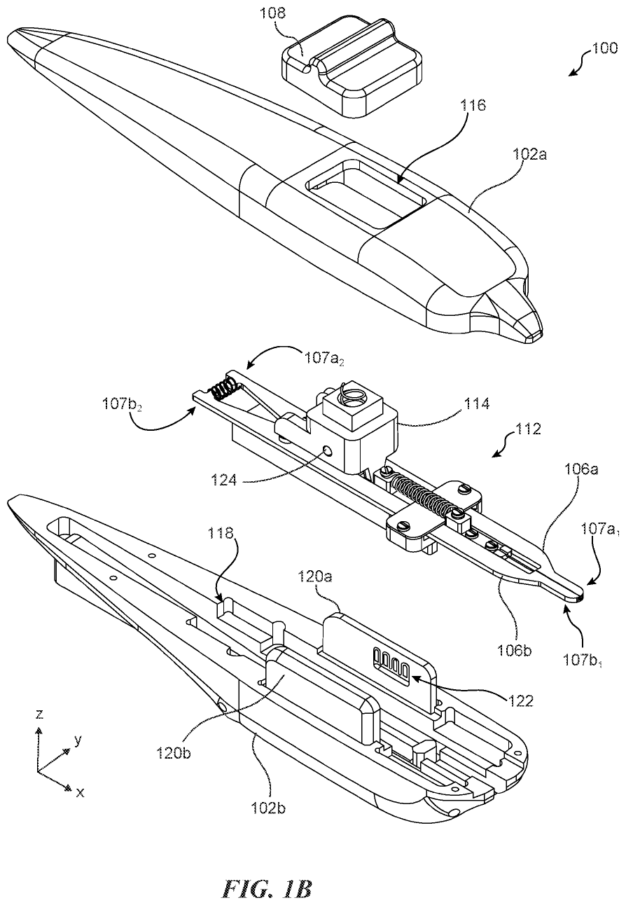

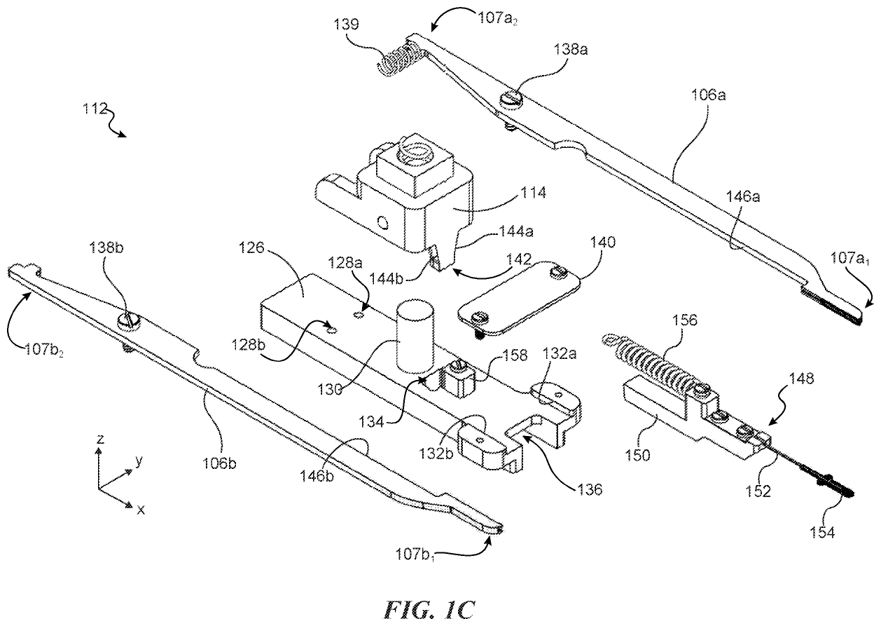

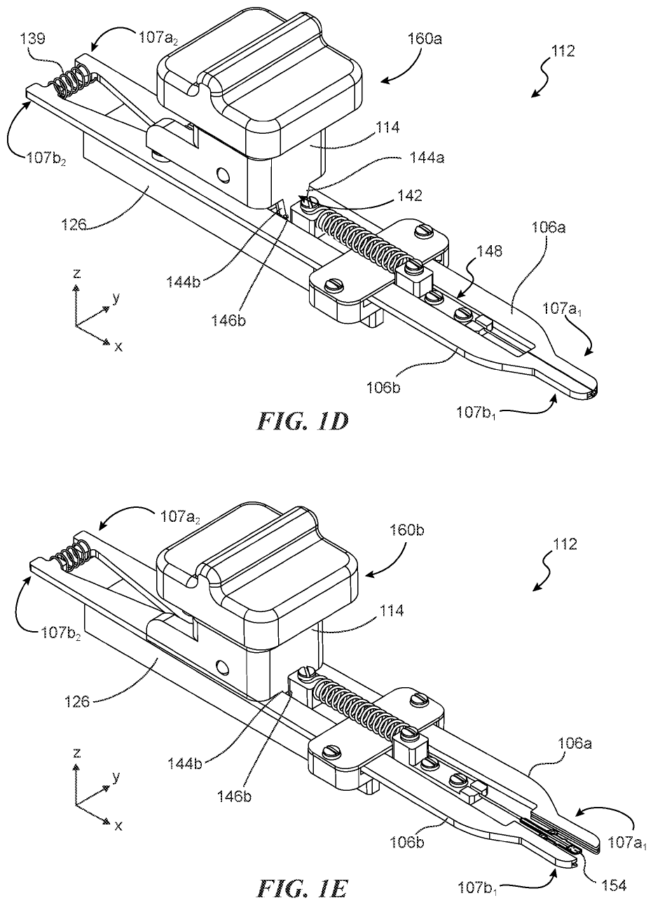

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the claims but are not described in detail with respect to FIGS. 1A-5E.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

As used herein, the use of relative terminology, such as “about”, “approximately”, “substantially” and the like refer to the stated value plus or minus ten percent. For example, the use of the term “about 100” refers to a range of from 90 to 110, inclusive. In instances in which the context requires otherwise and/or relative terminology is used in reference to something that does not include a numerical value, the terms are given their ordinary meaning to one skilled in the art.

Reference throughout this specification to the term “resistance” refers to fluid resistance unless the context clearly dictates otherwise. The terms “drainage rate” and “flow rate” are used interchangeably to describe the movement of fluid through a structure at a particular volumetric rate. The term “flow” is used herein to refer to the motion of fluid, in general.

Although certain embodiments herein are described in terms of shunting fluid from an anterior chamber of an eye, one of skill in the art will appreciate that the present technology can be readily adapted to shunt fluid from and/or between other portions of the eye (including the posterior chamber), or, more generally, from and/or between a first body region and a second body region of a patient. Moreover, while the certain embodiments herein are described in the context of glaucoma treatment, any of the embodiments herein, including those referred to as “glaucoma shunts” or “glaucoma devices” may nevertheless be used and/or modified to treat other diseases or conditions, including other diseases or conditions of the eye or other body regions of a patient. For example, the systems described herein can be used to treat diseases characterized by increased pressure and/or fluid build-up, including but not limited to heart failure (e.g., heart failure with preserved ejection fraction, heart failure with reduced ejection fraction, etc.), pulmonary failure, renal failure, hydrocephalus, and the like. Moreover, while generally described in terms of shunting aqueous, the systems described herein may be applied equally to shunting other fluid, such as blood or cerebrospinal fluid, between the first body region and the second body region.

FIG. 1A is a perspective view of a delivery system 100 (“system 100”) configured in accordance with embodiments of the present technology. The system 100 includes a housing 102, one or more forceps or arms 106 (individually identified as a first arm 106a and a second arm 106), a control member 108, and a trigger 110. The housing 102 has a first or distal end portion 104a and a second or proximal end portion 104b opposite the first end portion 104a. In the illustrated embodiment the housing 102 includes a first or upper housing portion 102a and a second or lower housing portion 102b. In other embodiments, the housing 102 can have more housing portions and/or be a single-piece component. Each of the arms 106 can extend distally from/beyond the distal end portion 104a of the housing 102, e.g., in a direction parallel or at least generally parallel to the x-axis, and can be configured to carry a shunt (not visible in FIG. 1A). As described in greater detail below, the control member 108 can be operably coupled to one or more of the arms 106 and configured to cause movement thereof. As will be explained in greater detail below, the trigger 110 can be configured to release/deploy the shunt from between the arms 106.

FIG. 1B is a partially-exploded perspective view of the system 100. As best seen in FIG. 1B, the system 100 further includes an actuation assembly 112. The actuation assembly 112 includes the arms 106 and an actuator 114. Each of the arms 106 can have a first or distal end portion 107a1, 107b1 and a second or proximal end portion 107a2, 107b2 opposite the first end portion 107a1, 107b1. The actuator 114 can be operably coupled to the control member 108, e.g., via a slot or opening 116 through the upper housing portion 102a. The actuation assembly 112 can be movably (e.g., slidably) disposed between the upper housing portion 102a and the lower housing portion 102b. In the illustrated embodiment, for example, the lower housing portion 102b includes a channel 118 and at least part of the actuation assembly 112 is movably positioned within the channel 118.

In some embodiments, the system 100 includes one or more movement guides 120 (individually identified as a first or left movement guide 120a and a second or right movement guide 120b) configured to control vertical (e.g., z-axis) and/or horizontal (e.g., x-axis, longitudinal, proximal/distal etc.) movement of at least part of the actuation assembly 112. In the illustrated embodiment, for example, both of the movement guides 120 include guide slots 122 (only one visible) configured to control vertical (e.g., z-axis) and/or horizontal (e.g., x-axis, longitudinal, proximal/distal, etc.) movement of at least part of the actuation assembly 112. The actuator 114 can include guide pegs 124 configured to be movably received within the guide slots 122, e.g., to control or otherwise direct vertical and/or horizontal movement of the actuator 114.

FIG. 1C is a partially exploded perspective view of the actuation assembly 112. For purposes of illustration and clarity, a number of other aspects of the system 100 are not shown. As best seen in FIG. 1C, the actuation assembly 112 can include a first base or carriage 126, which can include one or more ports 128 (individually identified as a first port 128a and a second port 128b), an actuator post 130, one or more pivot stops 132 (individually identified as a first or left pivot stop 132a and a second or right pivot stop 132b), a drive slot 134, and a channel 136. The ports 128 and the pivot stops 132 can be positioned proximate left and right sides of the first carriage 126. The actuator post 130 can extend upwardly/vertically (e.g., perpendicularly or at least generally perpendicularly) from the first carriage 126. The actuator post 130 and/or the drive slot 134 can be positioned between the ports 128 and the pivot stops 132. In the illustrated embodiment the actuator post 130 is positioned between the ports 128 and the drive slot 134 and the drive slot 134 is positioned between the actuator post 130 and the pivot stops 132. In other embodiments, the actuator post 130 and/or the drive slot 134 can have other suitable positions.

Each of the arms 106 can be pivotally coupled to the first carriage 126. In the illustrated embodiment, for example, the first arm 106a can be pivotally coupled to the first carriage 126 via a first coupling member 138a (e.g., a screw) configured to be received within the first port 128a and/or the second arm 106b can be pivotally coupled to the first carriage 126 via a second coupling member 138b (e.g., a screw) configured to be received within the second port 128b. The arms 106 can pivot relative to the first carriage 126 about their respective coupling members 138. A biasing element 139 (e.g., a coil spring) can be positioned between the arms 106, e.g., between the second end portions 107a2, 107b2 of the arms 106, to resist outward pivoting/rotation of the first end portions 107a₁, 107b₁ of the arms 106 and/or bias the first end portions 107a₁, 107b₁ of the arms 106 inwardly and toward one another.

When coupled to the first carriage 126, the arms 106 can be positioned inwardly from and/or between the pivot stops 132. In some embodiments, a retaining member or plate 140 can be coupled to the first carriage 126, such as in a position at least partially over the pivot stops 132, to hold the arms 106 between the pivot stops 132, e.g., during pivotal movement of the arms 106.

The actuator 114 can be movably coupled to the first carriage 126. In the illustrated embodiment, for example at least part of the actuator post 130 can be slidably received by the actuator 114 and configured to allow the actuator 114 to move vertically, e.g., along the actuator post 130 and/or in the z-axis direction, relative to the first carriage 126. In some embodiments the actuator 114 includes a drive post 142 having one or more drive surfaces 144 (individually identified as a first or left drive surface 144a and a second or right drive surface 144b). The drive post 142 can be aligned with the drive slot 134 such that downward movement of the actuator 114 (e.g., toward the first carriage 126) can position at least part of the drive post 142 within the drive slot 134. The drive surfaces 144 can be sloped/tapered. Accordingly, and as described in greater detail below, downward movement of the actuator 114 can cause the drive surfaces 144 to contact respective inner surfaces 146 of the arms 106 and thereby cause the arms 106 to pivot relative to the first carriage 126 until, e.g., the arms 106 contact the respective pivot stops 132.

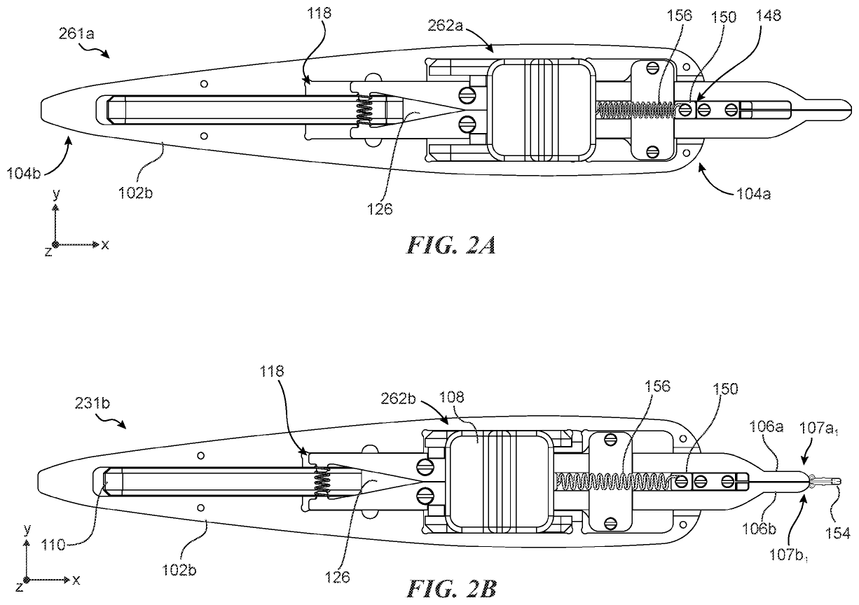

The actuation assembly 112 can further include a second base or carriage 148. The second carriage 148 can be movably coupled to the first carriage 126, e.g., via one or more biasing elements 156 which, in some embodiments, can be configured to bias the second carriage 148 toward the first carriage 126. The second carriage 148 can include a body 150 and a shunt carrying element 152 configured to be releasably coupled to a shunting system 154 (“shunt 154”), such as an adjustable shunting system. At least part of the body 150 can be movably positioned within the channel 136, e.g., to allow the second carriage 148 to move longitudinally (e.g., in a direction parallel, or at least generally parallel, to the x-axis) relative to the first carriage 126. At least part of the shunt carrying element 152 can be positioned within the shunt 154 and/or can be positioned to contact one or more internal components of the shunt 154. When carried by the shunt carrying element 152, at least part of the shunt 154 can be positioned between the first end portions 107a₁, 107b₁ of the arms 106. As described in greater detail below with reference to FIGS. 2A and 2B, at least the part of the shunt carrying element 152 can be removed from the shunt 154 in response to movement of the second carriage 148 relative to, e.g., the arms 106, to release the shunt 154 from the shunt carrying element 152.

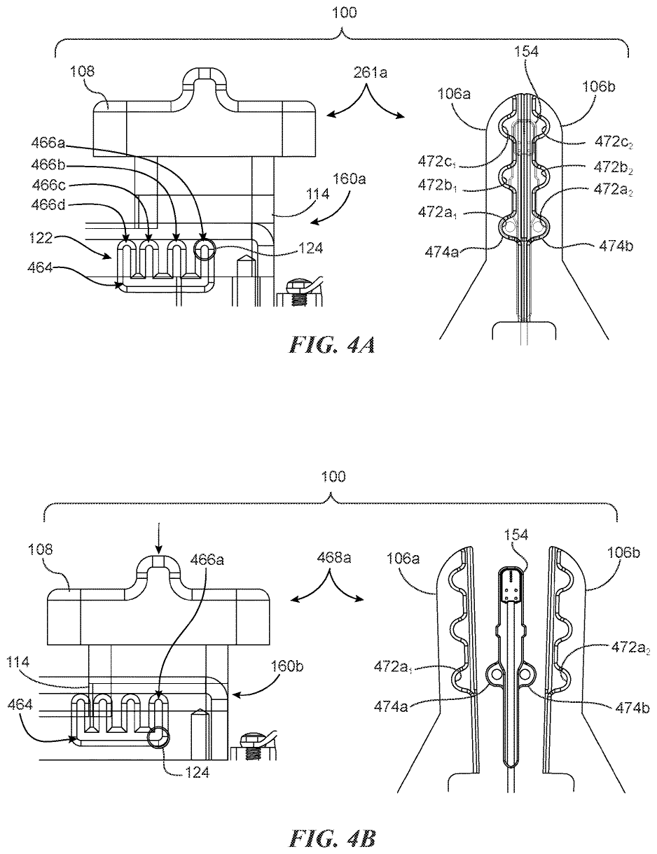

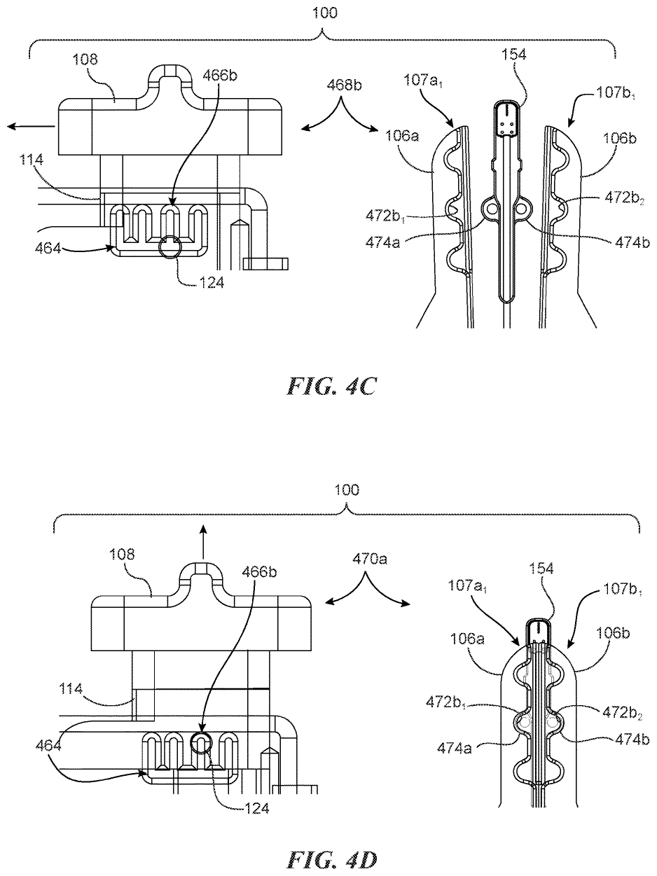

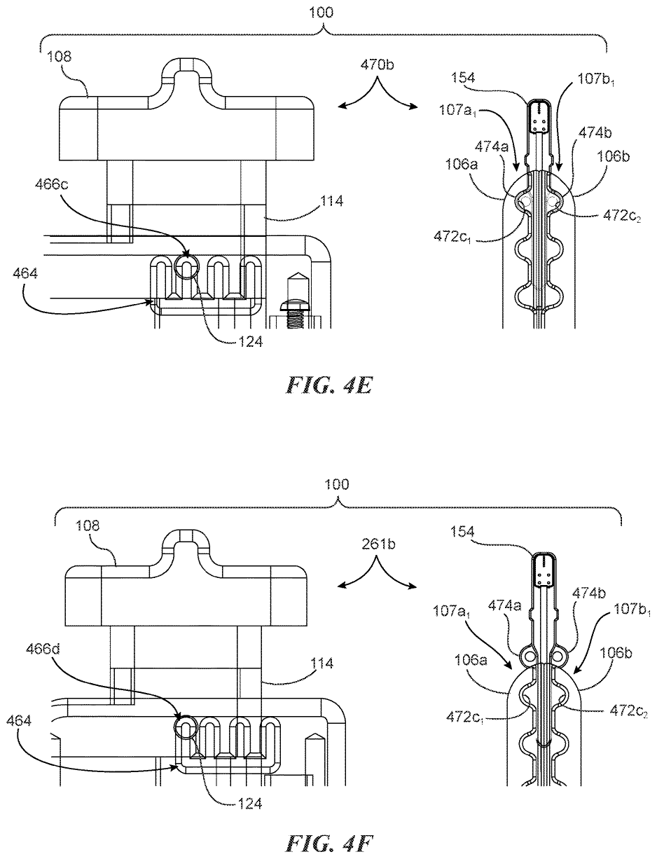

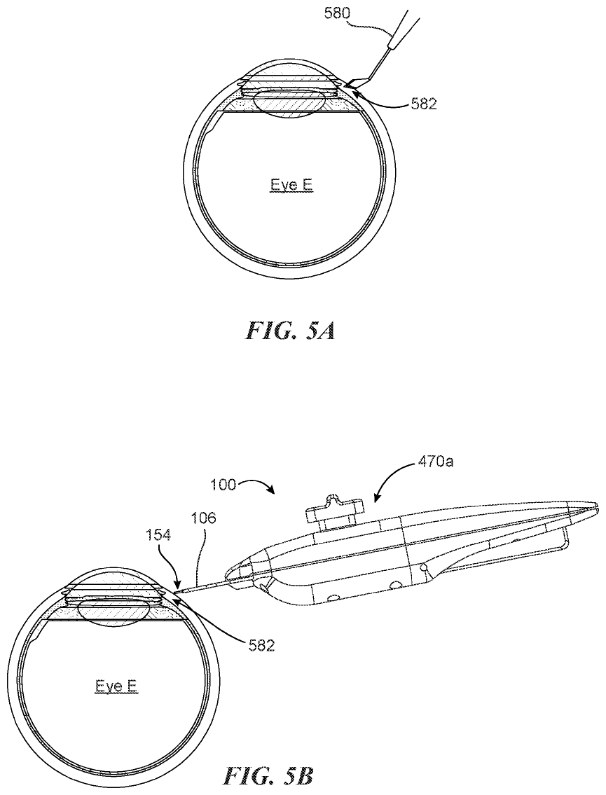

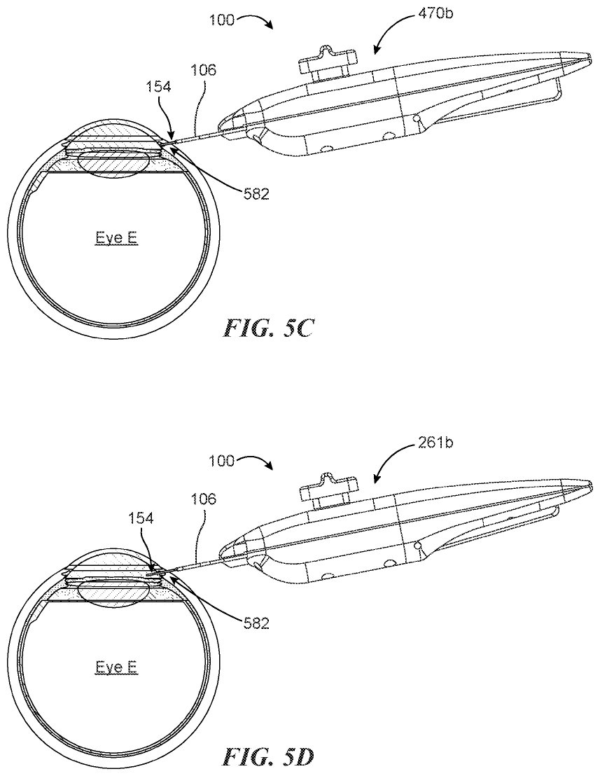

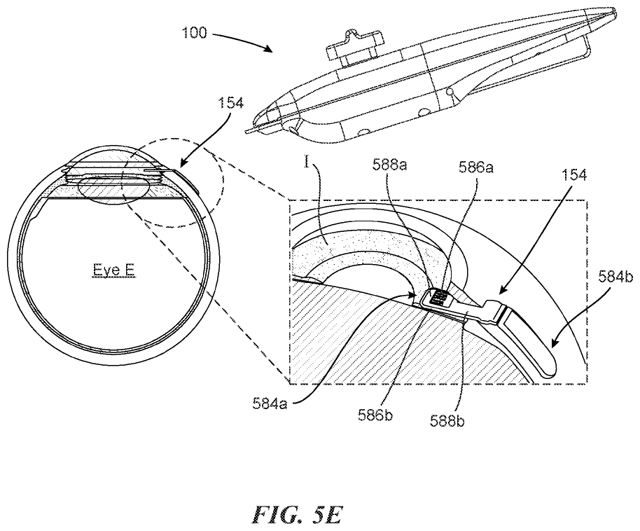

FIGS. 1D and 1E are perspective views illustration operation of the actuation assembly 112. In FIG. 1D, the actuator 114 is in a first position 160a and the biasing element 139 is biasing the first end portions 107a₁, 107b₁ of the arms 106 toward one another. In FIG. 1E, the actuator 114 has been moved toward the carriage 126 (e.g., vertically/downwardly) into a second position 160b in which the drive surfaces 144 (only the second drive surface 144b is visible) have been driven against the inner surfaces 146 (only the second inner surface 146b is identified) to cause the arms 106 to pivot relative to the carriage 126, e.g., to move the first end portions 107a₁, 107b₁ away from one another and/or to expose the shunt 154. Accordingly, the actuation assembly 112 can be configured such that vertical/downward movement of the actuator 114 (e.g., in a direction parallel, or at least generally parallel, to the z-axis) can cause pivotal movement of the arms 106 (e.g., in a plane coplanar with the x-and y-axes and/or normal to the x-axis). Positioning the shunt 154 between the arms 106 can at least partially protect the shunt 154 from damage, contamination, etc. As described in greater detail below with reference to FIGS. 4A-5E, actuating the actuation assembly 112 can expose varying lengths of the shunt 154 beyond the arms 106 to, e.g., allow the shunt 154 to be implanted in an eye of a patient.

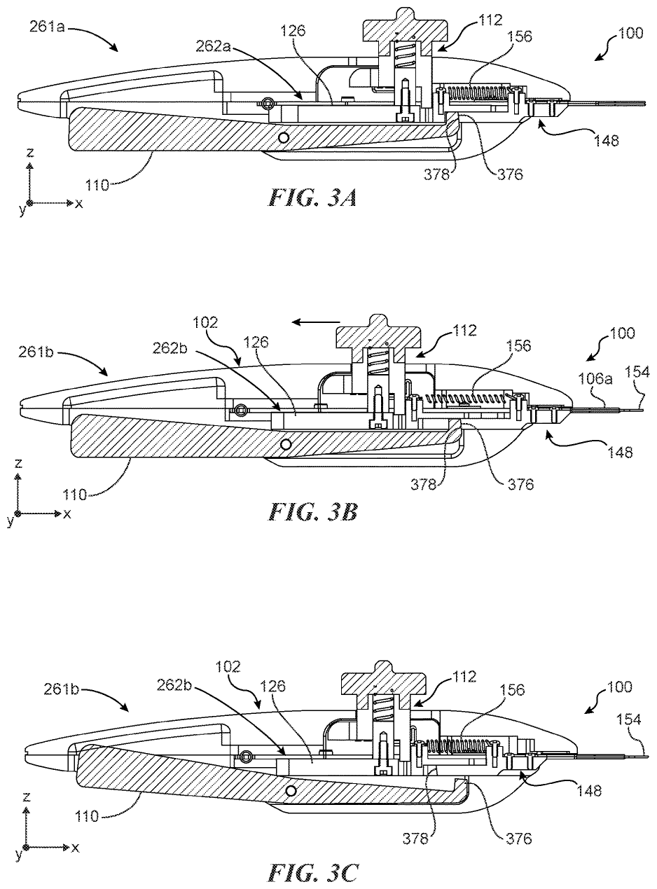

FIGS. 2A and 2B are perspective views of the system 100 with the upper housing portion 102a (FIG. 1A) omitted for illustrative clarity. The system 100 can be transitioned from a first configuration or state 261a (FIG. 2A) toward and/or to a second configuration or state 261b (FIG. 2B) in response to movement of, e.g., the first carriage 126. Referring to FIG. 2A, for example, the first carriage 126 is in a first position 262a within the channel 118, e.g., at or near the distal end portion 104a of the housing 102. In FIG. 2B, the first carriage 126 has been moved (e.g., by moving the control member 108) longitudinally/proximally in a direction parallel, or at least generally parallel, to the x-axis and to a second position 262b. In the second position 262b, the first carriage 126 can be positioned closer to the proximal end portion 104b of the housing 102 (compared to, e.g., the first position 262a of the first carriage 126). Because the arms 106 can be coupled to the first carriage 126, moving the first carriage 126 proximally can also move the arms 106 proximally, e.g., to cause at least part of the shunt 154 to be exposed distally beyond the distal end portions 107a₁, 107b₁ of the arms 106. As described in greater detail below with reference to FIGS. 3A-3C, the trigger 110 can contact the body 150 of the second carriage 148, e.g., to prevent or at least partially prevent movement of the second carriage 148 during the movement of the actuation assembly 112. Accordingly, as shown in FIG. 2B, moving the actuation assembly 112 proximally can lengthen/tension the biasing element 156 coupling the second carriage 148 to the first carriage 126.

FIGS. 3A-3C are side cross-sectional views of the system 100 illustrating movement of the actuation assembly 112 in accordance with embodiments of the present technology. In FIG. 3A, the carriage 126 is in the first position 262a and the system 100 is in the first configuration 261a, described previously with reference to FIG. 2A. An engagement portion 376 of the trigger 110 can contact a stopping surface 378 of the second carriage 148.

Referring to FIG. 3B, when the first carriage 126 is moved (e.g., distally) to the second position 262b and the system 100 is transitioned to the second configuration 261b, the contact between the engagement portion 376 and the stopping surface 378 can prevent, or at least partially prevent, movement of the second carriage 148 relative to, e.g., the housing 102. For example, the contact between the engagement portion 376 and the stopping surface 378 can allow the first carriage 126 to move, e.g., longitudinally/proximally in a direction parallel, or at least generally parallel, to the x-axis, without or substantially without changing a position of the second carriage 148 relative to the housing 102. This, in turn, can lengthen or tension the biasing element 156 coupling the second carriage 148 to the first carriage 126, as shown in FIG. 3B.

Referring to FIG. 3C, the trigger 110 can be rotated relative to the housing 102 to move the engagement portion 376 out of contact with the stopping surface 378. Rotating the trigger 110 after the system 100 has been transitioned to the second configuration 261b can allow the biasing element 156 to return toward and/or to its original (e.g., unlengthened, resting, etc.) configuration, thereby moving (e.g., pulling) the second carriage 148 toward the first carriage 126. The movement of second carriage 148 can, in turn, release the shunt 154 from the second carriage 148 by, e.g., withdrawing the shunt carrying element 152 (FIG. 1C) from within the shunt 154.

FIGS. 4A-4F each include a side view of the actuator 114 and a partially-transparent top view of a distal end portion of the arms 106 and illustrate movement of the actuator 114 and the arms 106 in accordance with embodiments of the present technology. Referring to FIG. 4A, the guide slots 122 can include a primary or horizontal guide slot 464 and one or more secondary or vertical guide slots 466 (individually identified as a first secondary slot 466a, a second secondary slot 466b, a third secondary slot 466c, and a fourth secondary slot 466d). The secondary guide slots 466 can be spaced apart along and extend (e.g., upwardly) from the primary guide slot 464. The guide peg 124 can be positioned within the guide slots 122, e.g., within individual ones of the primary slot 464 and/or the secondary slots 466, to guide movement of the actuator 114 relative to the guide slots 122. In at least some embodiments, for example, the guide peg 124 can move in a first direction (e.g., horizontally/longitudinally) through the primary guide slot 464 and in a second direction different than (e.g., perpendicular to) the first direction (e.g., vertically) through the second guide slots 466.

Each of the arms 106 can include one or more mating features 472 (individually identified as a first pair of mating features 472a₁₋₂, a second pair of mating features 472b₁₋₂, and a third pair of mating features 472c₁₋₂) configured to receive at least a portion of the shunt 154. In the illustrated embodiment, for example, the shunt 154 includes one or more (e.g., one or more pairs of) projections or wings 474a,b and each of the projections 464a,b can be received within corresponding ones (e.g., a corresponding pair) of the mating features 472. When the system 100 is in the first configuration 261a, the projections 474a,b of the shunt 154 can be positioned within the first pair of mating features 472a₁₋₂. When the projections 464a,b are received within/by the mating features 472, the mating features 472 can be configured to prevent, or at least partially prevent, movement of the shunt 154 relative to the arms 106 or another portion of the system 100. In some embodiments, the interaction between the mating features 472 and the shunt 154 is expected to increase the stability of the shunt 154 during a procedure by, e.g., reducing or prevent unintentional movement of the shunt 154 relative to the system 100. Additionally, or alternatively, the mating features 472 can hold the shunt 154 in place relative to the arms 106 with varying lengths of the shunt 154 exposed beyond the arms 106 to, e.g., allow the shunt 154 to be inserted into an eye of a patient, as described in greater detail below with reference to FIGS. 5A-5E. The mating features 472 are optional components that may not be included in some embodiments.

In the embodiment illustrated in FIG. 4A, the system 100 is in the first configuration 261a when the guide peg 124 is positioned at an upper end of the first secondary guide slot 466a, e.g., opposite the primary slot 464. Moving the actuator 114 relative to the guide slots 122 by, e.g., moving the handle 108, can change a configuration of the system 100 by, e.g., causing the arms 106 to move relative to the shunt 154. As described in greater detail below with reference to FIGS. 4B-4F, moving the actuator 114 can cause the system 100 to transition from the first configuration 261a toward and/or to the second configuration 261b (FIG. 2B).

Referring to FIG. 4B, the actuator 114 can be pressed (e.g., downwardly as shown by the arrow) to cause the peg 124 to move through the first secondary guide slot 466a toward and/or into the primary guide slot 464. When the peg 124 is positioned in the primary guide slot 464 and aligned with the first secondary guide slot 466a, e.g., as shown in FIG. 4B, the system 100 can be in a first transition configuration or state 468a. Downward movement of the actuator 114 can cause the arms 106 to pivot open, e.g., outwardly away from the shunt 154, such that the projections 474a,b are positioned between the arms 106. In the illustrated embodiment, for example, the projections 474a,b are positioned between (e.g., not received within) the first pair of mating features 472a₁₋₂.

Referring to FIG. 4C, the actuator 114 can be moved (e.g., horizontally and/or proximally as shown by the arrow) to cause the peg 124 to move through the primary guide slot 464 toward and/or into alignment with the second secondary guide slot 466b. When the peg 124 is positioned in the primary guide slot 464 and aligned with (e.g., positioned beneath) the second secondary guide slot 466b, e.g., as shown in FIG. 4C, the system 100 can be in a second transition configuration or state 468b. Horizontal movement of the actuator 114 can cause a corresponding horizontal movement of the arms 106. In at least some embodiments, for example, the proximal movement of actuator 114 shown in FIG. 4C can cause a corresponding proximal movement of the arms 106 relative to, e.g., the shunt 154. Accordingly, in the second transition configuration, the projections 474a,b can be positioned between the second pair of mating features 472b₁₋₂, and/or at least a portion of the shunt 154 can be positioned (e.g., distally) beyond the first end portions 107 of the arms 106, as shown in FIG. 4C.

Referring to FIG. 4D, the actuator 114 can be moved to cause the peg to move through the second secondary guide slot 466b toward, e.g., an end of the second secondary guide slot 466b opposite the primary slot 464, to transition the system 100 toward and/or to a first intermediate configuration or state 470a. The first intermediate configuration 470a can be a configuration of the system 100 between the first configuration 261a (FIG. 2A) and the second configuration 261b (FIG. 2B). Accordingly, transitioning the system 100 from the first configuration 261a to the second configuration 261b can include transitioning the system to the first intermediate configuration 470a.

In some embodiments, the actuator 114 can be biased upwardly by, e.g., a spring and/or other suitable biasing elements. In such embodiments, when the peg 124 is aligned with the second secondary guide slot 466b, the upward biasing of the actuator 114 can cause the peg 124 to move upwardly into the second secondary guide slot 466b. The upward movement of the actuator 114 can cause the arms 106 to be pivoted closed, e.g., inwardly toward the shunt 154, such that the projections 474a,b are positioned within the second pair of mating features 472b1-2 and/or at least a portion of the shunt 154 can be exposed beyond the first end portions 107 of the arms 106.

Referring to FIG. 4E, in some embodiments the system 100 can be transitioned to a second intermediate configuration or state 470b. In the illustrated embodiment, for example, the second intermediate configuration 470b is between the first intermediate configuration 470a (FIG. 4D) and the second configuration (FIG. 2B). The process of transitioning the system 100 to the second intermediate configuration 470b can be at least generally similar or identical to the process of transitioning the system 100 to the first intermediate configuration 470a described previously with reference to FIGS. 4A-4D except that, in the second intermediate configuration 470b, the peg 124 is aligned with/positioned in the third secondary guide shaft 466c. In the second intermediate configuration 470b, the projections 474a, b can be positioned within the third pair of mating features 472c1-2 and/or at least a portion of the shunt 154 can be exposed beyond the first end portion 107 of the arms 106.

Referring to FIG. 4F, when the system 100 is in the second configuration 261b, the peg 124 can be positioned in the fourth secondary guide shaft 466d. The process of transitioning the system 100 to the second configuration 261b can be at least generally similar or identical to the process of transitioning the system 100 to the first intermediate configuration 470a described previously with reference to FIGS. 4A-4D except that, in the second configuration 261b, the peg 124 is aligned with/positioned in the fourth secondary guide shaft 466d. In the second configuration 261b, the projections 474a, b can be positioned distally beyond the third pair of mating features 472c1-2, e.g., distally beyond the first end portions 107 of the arms 106.

FIGS. 5A-5E illustrate various stages of an operation for deploying the shunt 154 into a patient's eye E using the system 100 in accordance with embodiments of the present technology. In a particular example, the system 100 can be used to deploy the shunt 154 such that, after implantation, the shunt 154 is positioned to route fluid from an anterior chamber of the patient's eye E to a suitable outflow location, such as a subconjunctival bleb space (e.g., to treat glaucoma).

Referring first to FIG. 5A, one or more tools 580 (e.g., a keratome) can be used to make one or more incisions 582 in the eye E. Referring next to FIG. 5B, the system 100 can be transitioned toward and/or to the first intermediate configuration 470a, as described above with reference to FIG. 4D. In the first intermediate configuration 470a, at least a portion of the shunt 154 can be exposed beyond the arms 106 of the system. The portion of the shunt 154 exposed beyond the arms can be inserted into the eye E through the incision 582.

Referring next to FIG. 5C, the system 100 can be transitioned toward and/or to the second intermediate configuration 470b, as described above with reference to FIG. 4E. In the second intermediate configuration 470b, more of the shunt 154 can be exposed distally beyond the arms 106 of the system 100. Accordingly, transitioning the system 100 toward and/or to the second intermediate configuration 470b (e.g., from the first intermediate configuration described above) can position the shunt 154 further (e.g., deeper) within the eye E.

Referring to FIG. 5D, the system 100 can be transitioned toward and/or to the second configuration 261b, as described above with reference to FIGS. 2B and 4F. In the second configuration 261b, a greater/increased length of the shunt 154 can be exposed beyond the arms 106 of the system, e.g., greater than the length in the second intermediate configuration 470b (FIG. 5C). Accordingly, transitioning the system 100 toward and/or to the second configuration 261b (e.g., from the first intermediate configuration described above) can position the shunt 154 further (e.g., deeper) within the eye E, e.g., compared to when the system 100 is in the second intermediate configuration 470b (FIG. 5C). Additionally, transitioning the system 100 toward and/or to the second configuration 261b can position the projections 474a,b (FIG. 4F) of the shunt 154 distally of the arms 106 and thereby allow the shunt 154 to be uncoupled from the system 100. For example, as shown in FIG. 5D and described previously with reference to FIGS. 4A-4C, the trigger 110 can be pressed to release the shunt 154 from the system 100 by, e.g., withdrawing the shunt carrying element 152 (FIG. 1C) from within the shunt 154.

Referring to FIG. 5E, the system 100 can be withdrawn to leave the shunt 154 implanted within the eye E. The shunt 154 can be positioned such that a first or inflow portion 584a of the shunt 154 is positioned in a first body region and a second or outflow portion 584b of the shunt 154 is positioned in a second body region. In this way, after implantation, fluid within the first body region can flow/drain through the shunt 154 toward and/or into the second body region. In some embodiments, the shunt 154 can be configured to assume a curved, bent, or preformed shape during/after implantation in the patient, as shown in FIG. 5E. Additionally, or alternatively, the system 100 can be configured to adjust the shape of the system 100 in situ, and/or one or more of the tools 580 can be used to adjust the shape of the system 100 in situ.

Although in FIG. 5E the first portion 584a of the shunt 154 is illustrated as being positioned anterior to (e.g., in front of/above) the patient's iris I, in other embodiments the first portion 584a can be positioned posterior to (e.g., behind/below) the iris I (also referred to as “sub-iris” positioning). The sub-iris positioning of the shunt 154 is expected to reduce or prevent corneal endothelial disease and/or failure, for example, by reducing or preventing disruption to nutrient and/or other fluid and/or chemical transport to and/or through the corneal endothelium. In the illustrated embodiment, the shunt 154 is configured to receive fluid (e.g., aqueous) through one or more openings or inlets in an upper surface 588a of the shunt 154. In these and other embodiments, including when the shunt 154 has a sub-iris position, the shunt 154 can be configured to receive fluid through one or more lateral openings, e.g., positioned in one or more sides 586a, 586b of the shunt 154, and/or through one or more openings in a bottom surface 588b of the shunt 154.

In general, the shunt 154 can be configured to actuate and/or change its resistance to fluid flow in response to energy (e.g., laser energy) delivered from a source external to the patient. An example of such a configuration is described in detail in U.S. Patent App. Publication No. US 2021/0251806, filed Feb. 12, 2021, and incorporated herein by reference in its entirety for all purposes. In embodiments in which the shunt 154 is positioned sub-iris, the shunt 154 can be actuated using a number of techniques. For example, a portion of the iris I can be removed (e.g., iridectomy) to provide line-of-sight access to the shunt 154. Additionally, or alternatively, the shunt 154 can be positioned such that at least a portion of the shunt 154 (e.g., at least part of the first portion 584a) can be exposed when the eye E undergoes pupil dilation. In such embodiments, the pupil of the eye E can be dilated and then energy can be delivered to actuate the shunt 154. In these and other embodiments, the energy can be targeted using a first energy source (e.g., a first laser) configured to transmit first energy (e.g., targeting energy) at or near a first wavelength to which the iris I is translucent or transparent, and then a same or different energy source can transmit second energy (e.g., actuating energy) at a second wavelength different than the first wavelength.

In some embodiments, the shunt 154 can be configured to reduce or prevent cellular growth onto, over, and/or around at least a portion of the shunt 154. In at least some embodiments, for example, all or a portion of the shunt 154 can include one or more radioisotopes configured to inhibit or prevent cellular growth. The radioisotopes can include Phosporous-32, Strontium-89, Strontium-90, Yttrium-90, and/or another suitable radioisotope. Individual ones of the radioisotopes can emit alpha, beta, and/or gamma radiation, each of which are expected to inhibit or prevent cellular growth on, over, and/or near the shunt 154. For a given patient, the radioisotope(s) used with the shunt 154 can be selected based at least partially on the half-life of the radioisotope, the type of radiation, and/or the energy of the emitted alpha, beta, and/or gamma particles. The radioisotopes can be naturally-occurring or manufactured (e.g., using a cyclotron, reactor-produced, etc.).

### EXAMPLES

Several aspects of the present technology are set forth in the following examples:

1. A delivery system for implanting a shunt in an eye of a patient, the delivery system comprising:
a housing;
a pair of movement guides positioned within the housing; and
an actuation assembly positioned at least partially between the pair of movement guides,
   wherein the actuation assembly includes
   an actuator, and
   a pair of arms operably engaged by the actuator,
wherein
   the actuation assembly is configured to releasably carry a shunt at least partially between the pair of arms, and
   the actuator is movable relative to the pair of movement guides to cause movement of the pair of arms relative to the housing.

2. The delivery system of example 1 wherein the actuation assembly is configured such that movement of the actuator in a first direction causes pivotal movement of the pair of arms.

3. The delivery system of example 2 wherein the first direction is perpendicularly toward or away from the pair of arms.

4. The delivery system of any of examples 1-3 wherein the actuator includes a pair of angled drive surfaces, and wherein individual ones of the pair of angled drive surfaces face a corresponding inner surface of one of the pair of arms.

5. The delivery system of any of examples 1-4 wherein the actuation assembly is configured such that movement of the actuator in a second direction relative to the pair of movement guides causes movement of the pair of arms relative to the housing in the second direction.

6. The delivery system of example 5 wherein the second direction is parallel to a longitudinal axis of the housing.

7. The delivery system of any of examples 1-6 wherein the actuation assembly further comprises:
a first carriage including the actuator and the pair of arms; and
a second carriage configured to releasably receive the shunt,
wherein
   the first carriage and the second carriage are movably positioned within the housing, and
   the second carriage is movably coupled to the first carriage by a biasing element.

8. The delivery system of example 7 further comprising a trigger operably coupled to the housing and pivotable between (i) a first position in which the trigger contacts the second carriage to at least partially prevent movement of the second carriage relative to the first carriage and (ii) a second position in which the trigger allows the biasing element to move the second carriage toward the first carriage.

9. The delivery system of any of examples 1-8 wherein the actuator is movable relative to the pair of movement guides to transition the delivery system between (i) a first configuration in which the shunt is positioned between the pair of arms and (ii) a second configuration in which at least a portion of the shunt is exposed beyond end portions of the pair of arms.

10. The delivery system of example 9 wherein:
in the first configuration, the pair of arms have a first position relative to the housing; and
in the second configuration, the pair of arms have a second position different than the first position.

11. The delivery system of example 10 wherein the second position is proximal of the first position, and wherein at least the portion of the shunt is exposed distally beyond distal end portions of the pair of arms.

12. The delivery system of any of examples 1-11 wherein individual ones of the pair of arms include one or more mating features configured to releasably receive corresponding mating portions of the shunt to at least partially prevent movement of the shunt relative to the pair of arms.

13. The delivery system of example 12 wherein the mating features include recesses, and wherein the mating portions of the shunt include projections configured to be positioned at least partially within individual ones of the recesses.

14. The delivery system of example 12 wherein:
the mating features include a first pair of mating features and a second pair of mating features;
the shunt has a first position relative to the pair of arms when the mating portions of the shunt are received by the first pair of mating features; and
the shunt has a second position relative to the pair of arms, different than the first position, when the mating portions of the shunt are received by the second pair of mating features.

15. The delivery system of example 14 wherein, in the first position, a first length of the shunt is exposed beyond the pair of arms, and wherein, in the second position, a second length of the shunt greater than the first length is exposed beyond the pair of arms.

16. The delivery system of any of examples 1-15 wherein the shunt is an adjustable shunting system for treating glaucoma of the patient.

17. An implantable shunt delivery system, comprising:
a housing;
a first carriage positioned within the housing;
a pair of arms pivotally coupled to the first carriage;
a second carriage positioned within the housing and movably coupled to the first carriage via a biasing element, wherein the second carriage is configured to releasably carry a shunt and position the shunt at least partially between the pair of arms; and
a trigger coupled to the housing and configured to be movable between (i) a first position in which the trigger contacts the second carriage to at least partially prevent movement of the second carriage relative to the first carriage and (ii) a second position in which the trigger allows the biasing element to move the second carriage toward the first carriage to release the shunt from the second carriage.

18. The implantable shunt delivery system of example 17, further comprising an actuator operably coupled to the pair of arms, wherein movement of the actuator in a first direction causes the pair of arms to pivot open, and wherein movement of the actuator in a second direction opposite the first direction causes the pair of arms to pivot closed.

19. The implantable shunt delivery system of example 17 or example 18 wherein movement of the actuator in a third direction causes the pair of arms to move in the third direction, and wherein movement of the actuator in a fourth direction opposite the third direction causes the pair of arms to move in the fourth direction.

20. The implantable shunt delivery system of example 19 wherein the actuator is configured to move in the third direction after the actuator has been moved in the first direction to cause the pair of arms to pivot open.

21. The implantable shunt delivery system of examples 17-20 wherein the first carriage is configured to move relative to the housing and independently of the second carriage.

### CONCLUSION

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, any of the features of the adjustable shunts described herein may be combined with any of the features of the other adjustable shunts described herein and vice versa. Moreover, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions associated with intraocular shunts have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A delivery system for implanting a shunt in an eye of a patient, the delivery system comprising:
a housing;
a pair of movement guides positioned within the housing; and
an actuation assembly positioned at least partially between the pair of movement guides,
wherein the actuation assembly includes
an actuator, and
a pair of arms operably engaged by the actuator,
wherein
the actuation assembly is configured to releasably carry a shunt at least partially between the pair of arms, and
the actuator is movable relative to the pair of movement guides to cause movement of the pair of arms relative to the housing.

2. The delivery system of claim 1 wherein the actuation assembly is configured such that movement of the actuator in a first direction causes pivotal movement of the pair of arms.

3. The delivery system of claim 2 wherein the first direction is perpendicularly toward or away from the pair of arms.

4. The delivery system of claim 1 wherein the actuator includes a pair of angled drive surfaces, and wherein individual ones of the pair of angled drive surfaces face a corresponding inner surface of one of the pair of arms.

5. The delivery system of claim 1 wherein the actuation assembly is configured such that movement of the actuator in a second direction relative to the pair of movement guides causes movement of the pair of arms relative to the housing in the second direction.

6. The delivery system of claim 5 wherein the second direction is parallel to a longitudinal axis of the housing.

7. The delivery system of claim 1 wherein the actuation assembly further comprises:
a first carriage including the actuator and the pair of arms; and
a second carriage configured to releasably receive the shunt,
wherein
the first carriage and the second carriage are movably positioned within the housing, and
the second carriage is movably coupled to the first carriage by a biasing element.

8. The delivery system of claim 7 further comprising a trigger operably coupled to the housing and pivotable between (i) a first position in which the trigger contacts the second carriage to at least partially prevent movement of the second carriage relative to the first carriage and (ii) a second position in which the trigger allows the biasing element to move the second carriage toward the first carriage.

9. The delivery system of claim 1 wherein the actuator is movable relative to the pair of movement guides to transition the delivery system between (i) a first configuration in which the shunt is positioned between the pair of arms and (ii) a second configuration in which at least a portion of the shunt is exposed beyond end portions of the pair of arms.

10. The delivery system of claim 9 wherein:
in the first configuration, the pair of arms have a first position relative to the housing; and
in the second configuration, the pair of arms have a second position different than the first position.

11. The delivery system of claim 10 wherein the second position is proximal of the first position, and wherein at least the portion of the shunt is exposed distally beyond distal end portions of the pair of arms.

12. The delivery system of claim 1 wherein individual ones of the pair of arms include one or more mating features configured to releasably receive corresponding mating portions of the shunt to at least partially prevent movement of the shunt relative to the pair of arms.

13. The delivery system of claim 12 wherein the mating features include recesses, and wherein the mating portions of the shunt include projections configured to be positioned at least partially within individual ones of the recesses.

14. The delivery system of claim 12 wherein:
the mating features include a first pair of mating features and a second pair of mating features;
the shunt has a first position relative to the pair of arms when the mating portions of the shunt are received by the first pair of mating features; and
the shunt has a second position relative to the pair of arms, different than the first position, when the mating portions of the shunt are received by the second pair of mating features.

15. The delivery system of claim 14 wherein, in the first position, a first length of the shunt is exposed beyond the pair of arms, and wherein, in the second position, a second length of the shunt greater than the first length is exposed beyond the pair of arms.

16. The delivery system of claim 1 wherein the shunt is an adjustable shunting system for treating glaucoma of the patient.

17. An implantable shunt delivery system, comprising:
a housing;
a first carriage positioned within the housing;
a pair of arms pivotally coupled to the first carriage;
a second carriage positioned within the housing and movably coupled to the first carriage via a biasing element, wherein the second carriage is configured to releasably carry a shunt and position the shunt at least partially between the pair of arms; and
a trigger coupled to the housing and configured to be movable between (i) a first position in which the trigger contacts the second carriage to at least partially prevent movement of the second carriage relative to the first carriage and (ii) a second position in which the trigger allows the biasing element to move the second carriage toward the first carriage to release the shunt from the second carriage.

18. The implantable shunt delivery system of claim 17, further comprising an actuator operably coupled to the pair of arms, wherein movement of the actuator in a first direction causes the pair of arms to pivot open, and wherein movement of the actuator in a second direction opposite the first direction causes the pair of arms to pivot closed.

19. The implantable shunt delivery system of claim 17 wherein movement of the actuator in a third direction causes the pair of arms to move in the third direction, and wherein movement of the actuator in a fourth direction opposite the third direction causes the pair of arms to move in the fourth direction.

20. The implantable shunt delivery system of claim 18 wherein the actuator is configured to move in the third direction after the actuator has been moved in the first direction to cause the pair of arms to pivot open.

21. The implantable shunt delivery system of claim 17 wherein the first carriage is configured to move relative to the housing and independently of the second carriage.
